(19) United States
(12) Patent Application Publication
(10) Pub. No.: US 2024/0237936 A1
(43) Pub. Date: Jul. 18, 2024

(54) MYOELECTRIC SENSOR

(71) Applicant: SHINKO ELECTRIC INDUSTRIES CO., LTD., Nagano (JP)

(72) Inventor: Kenichi MORI, Nagano (JP)

(21) Appl. No.: 18/408,935

(22) Filed: Jan. 10, 2024

(30) Foreign Application Priority Data
Jan. 13, 2023 (JP) 2023-003928

(51) Int. Cl. A61B 5/389; A61B 5/00

(52) U.S. Cl. CPC A61B 5/389; A61B 5/6833; A61B 2562/164

(57) ABSTRACT

A myoelectric sensor includes a wearable portion configured to be worn on a living body and deformable in response to deformation of the living body, an electrode sheet disposed on a surface of the wearable portion, and fixing members configured to fix the electrode sheet to the surface, wherein the electrode sheet includes a plurality of bioelectrode units, and interconnect members each electrically connecting two bioelectrode units among the plurality of bioelectrode units, wherein the interconnect members are located between the wearable portion and the fixing members, and the fixing members press the interconnect members to the surface.

### CROSS-REFERENCE TO RELATED APPLICATIONS

The present application is based upon and claims the benefit of priority from the prior Japanese Patent Application No. 2023-003928 filed on Jan. 13, 2023, with the Japanese Patent Office, the entire contents of which are incorporated herein by reference.

### FIELD

The disclosures herein relate to myoelectric sensors.

### BACKGROUND

Some myoelectric sensors have a plurality of bioelectrode units connected to each other to serve as a device for detecting the movement of a muscle of a living body.

Today, it is desired for myoelectric sensors to have more improved detection accuracy.

Accordingly, there may be a need for a myoelectric sensor with improved detection accuracy.

### RELATED-ART DOCUMENT

### Patent Document

[Patent Document 1] Japanese Laid-Open Patent Publication No. 2018-023568

[Patent Document 2] Japanese Patent No. 5184520

[Patent Document 3] Japanese Laid-Open Patent Publication No. 2011-161025

### SUMMARY

According to an aspect of the embodiment, a myoelectric sensor includes a wearable portion configured to be worn on a living body and deformable in response to deformation of the living body, an electrode sheet disposed on a surface of the wearable portion, and fixing members configured to fix the electrode sheet to the surface, wherein the electrode sheet includes a plurality of bioelectrode units, and interconnect members each electrically connecting two bioelectrode units among the plurality of bioelectrode units, wherein the interconnect members are located between the wearable portion and the fixing members, and the fixing members press the interconnect members to the surface.

The object and advantages of the embodiment will be realized and attained by means of the elements and combinations particularly pointed out in the claims. It is to be understood that both the foregoing general description and the following detailed description are exemplary and explanatory and are not restrictive of the invention, as claimed.

### BRIEF DESCRIPTION OF DRAWINGS

FIGS. 1A and 1B are drawings illustrating an example of a myoelectric sensor according to a first embodiment;

FIG. 2 is a cross-sectional view illustrating an example of a bioelectrode unit;

FIG. 3 is a drawing illustrating an example of a method of using the myoelectric sensor according to the first embodiment;

FIG. 4 is a plan view illustrating an example of a myoelectric sensor according to a second embodiment;

FIG. 5 is a plan view illustrating an example of a myoelectric sensor according to a third embodiment;

FIG. 6 is a plan view illustrating an example of a myoelectric sensor according to a fourth embodiment;

FIG. 7 is a plan view illustrating an example of a myoelectric sensor according to a fifth embodiment; and

FIG. 8 is a plan view illustrating an example of a myoelectric sensor according to a sixth embodiment.

### DESCRIPTION OF EMBODIMENTS

In the following, embodiments of the present invention will be described with reference to the accompanying drawings.

In the following, embodiments of the present disclosure will specifically be described with reference to the accompanying drawings. In the instant specification and drawings, elements having substantially the same functional configuration may be referred to by the same reference numerals, and a duplicate description thereof may be omitted.

### First Embodiment

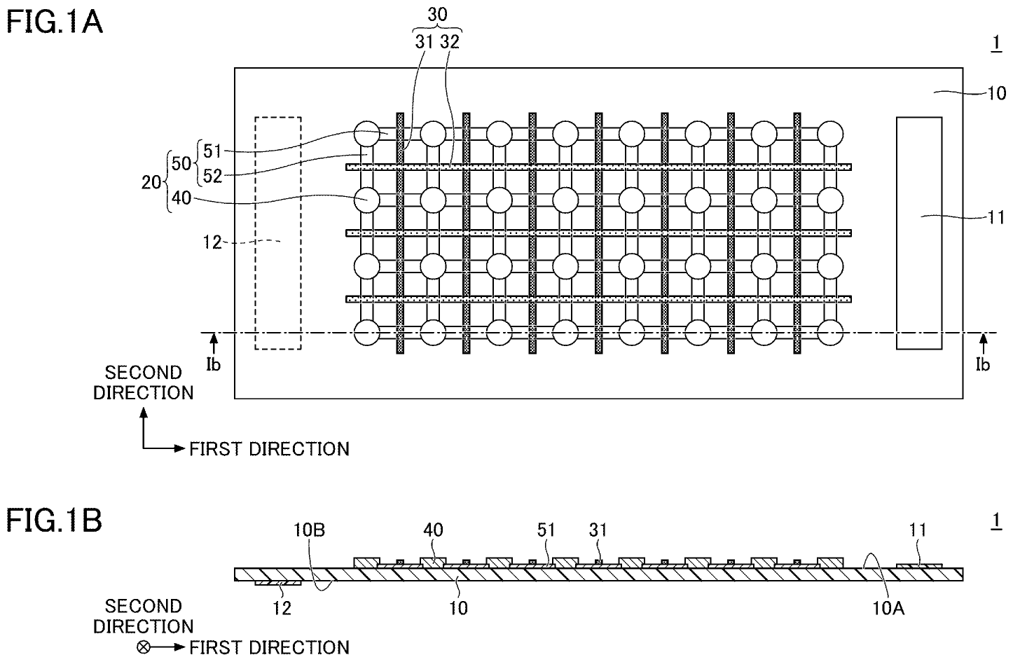

In what follows, the first embodiment will be described. The first embodiment relates to a myoelectric sensor. FIGS. 1A and 1B are drawings illustrating an example of a myoelectric sensor according to the first embodiment. FIG. 1A is a plan view, and FIG. 1B is a cross-sectional view. FIG. 1B corresponds to a cross-sectional view taken along the line Ib-Ib in FIG. 1A.

The myoelectric sensor 1 according to the first embodiment includes a wearable portion 10, an electrode sheet 20, and fixing members 30.

The wearable portion 10 has elasticity. The wearable portion 10 is worn on a living body. The wearable portion 10 is deformable to conform to the deformation of the living body. The wearable portion 10 is, for example, a supporter made of urethane. The wearable portion 10 includes a pair of fasteners 11 and 12 that are secured to each other and are wrapped around an arm, a leg or a waist of a living body, for example. The fastener 11 is disposed on one surface 10A of the wearable portion 10, and the fastener 12 is disposed on the other surface 10B. Alternatively, the entirety of the surface 10A of the wearable portion 10 may be a loop fastener, and the fastener 12 may be a hook fastener. The wearable portion 10 has a longitudinal direction and a transverse direction perpendicular to the longitudinal direction. The fasteners 11 and 12 are disposed apart from each other in the longitudinal direction.

The electrode sheet 20 is disposed on the surface 10A of the wearable portion 10. The electrode sheet 20 includes a plurality of bioelectrode units 40 and a plurality of interconnect members 50 each of which electrically connects two bioelectrode units 40 among the plurality of bioelectrode units 40.

The bioelectrode units 40 are arranged in rows and columns, each row extending in a first direction and each column extending in a second direction perpendicular to the first direction. The first direction is parallel to the longitudinal direction of the wearable portion 10, and the second direction is parallel to the transverse direction of the wearable portion 10. The second direction is a direction rotated 90 degrees counterclockwise relative to the first direction. The bioelectrode units 40 are arranged at the nodes of a grid, and preferably arranged at the nodes of a square grid.

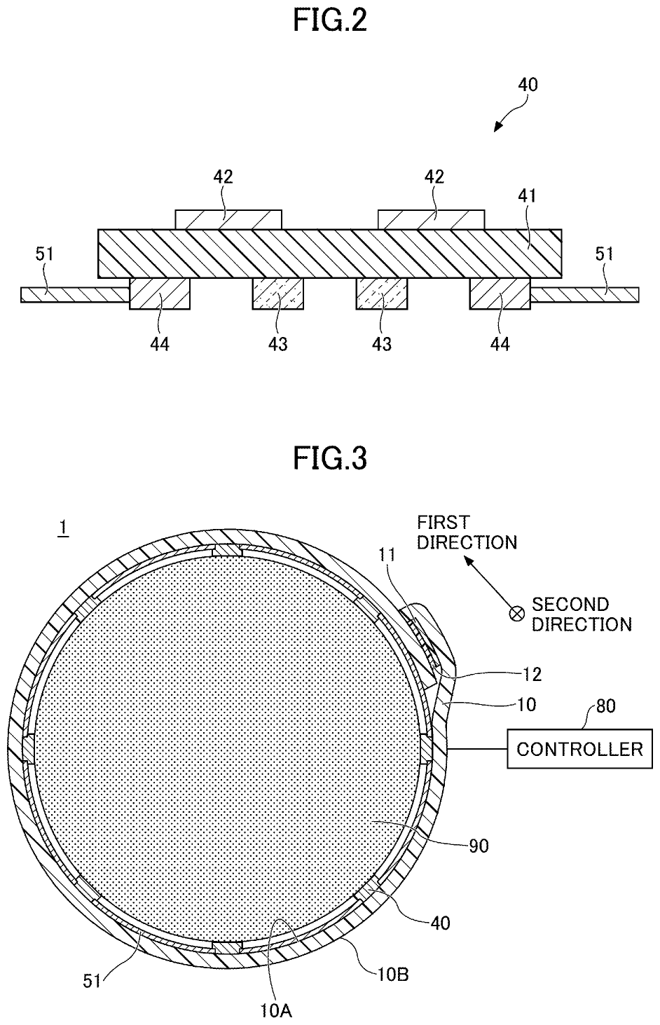

FIG. 2 is a cross-sectional view illustrating an example of a bioelectrode unit 40. The bioelectrode unit 40 includes a substrate 41, a pair of myoelectric electrodes 42, electronic components 43, and connectors 44. The electronic components 43 and the connectors 44 are disposed, for example, on one surface of the substrate 41, and the myoelectric electrodes 42 are disposed on the other surface of the substrate 41. The number of electronic components 43 is not limited to a particular number. The electronic components 43 constitute a signal processing circuit. The signal processing circuit includes, for example, a filter, a differential amplifier, and a switch. The connectors 44 are electrically connected to the electronic components 43. The interconnect members 50, which will be described later, are connected to the connectors 44. The myoelectric electrodes 42 are electrically connected to the electronic components 43. The signal processing circuit constituted by the electronic components 43 performs signal processing on electrical signals such as a current flowing between the pair of myoelectric electrodes 42. The bioelectrode unit 40 may sometimes be referred to as an active electrode.

Each interconnect member 50 electrically connects two bioelectrode units 40 to each other. The interconnect member 50 is, for example, a flexible printed circuit board, which includes a plurality of flexible resin insulating layers and one or more metal interconnect layers such as copper foils disposed between the insulating layers. The plurality of interconnect members 50 include a plurality of first interconnect members 51 and a plurality of second interconnect members 52. The first interconnect members 51 each electrically connect two bioelectrode units 40 situated next to each other in the first direction. The second interconnect members 52 each electrically connect two bioelectrode units 40 situated next to each other in the second direction. The first interconnect members 51 extend in the first direction, and the second interconnect members 52 extend in the second direction. The first interconnect members 51 and the second interconnect members 52 are arrayed in the first and second directions to form a grid.

The fixing members 30 fix the electrode sheet 20 to the surface 10A of the wearable portion 10. The fixing members 30 are made of, for example, urethane. The fixing members 30 press the interconnect members 50 against the surface 10A. In the case of the entire surface 10A being a loop fastener, the fixing members 30 may be a hook fastener. The fixing members 30 include first fixing members 31 and second fixing members 32. The first fixing members 31 secure the first interconnect members 51 arrayed in the second direction to the surface 10A. The second fixing members 32 secure the second interconnect members 52 arrayed in the first direction to the surface 10A.

In the following, a method of using the myoelectric sensor 1 will be described. FIG. 3 is a drawing illustrating an example of a method of using the myoelectric sensor 1 according to the first embodiment.

As illustrated in FIG. 3, the myoelectric sensor 1 is attached to a living body such that the myoelectric electrodes 42 of each bioelectrode unit 40 is in contact with the skin. For example, the myoelectric sensor 1 is attached to an arm 90 of a human body. In this case, the myoelectric sensor 1 is wrapped around the arm 90 such that the transverse direction of the wearable portion 10 is substantially parallel to the muscle fibers to be measured, and the fasteners 11 and 12 are fixed to each other. The myoelectric sensor 1 is connected to a controller 80 by wire or radio. In the case of wire connection, for example, one or more bioelectrode units 40 arranged at outermost grid nodes in the second direction, among the grid nodes constituted by the plurality of bioelectrode units 40, are connected to the controller 80 by cable or the like. In the case of wireless connection, for example, one of the bioelectrode units 40 may be equipped with a wireless transceiver, or the myoelectric sensor 1 is connected to a wireless transceiver.

Power is supplied to the myoelectric sensor 1 from the controller 80 through the interconnect members 50. Under the control of the controller 80, a given bioelectrode unit 40 detects, via the myoelectric electrodes 42, an electrical signal generated in response to the movement of a muscle such as a muscle contraction, and outputs the signal processed by the signal processing circuit to the controller 80 via one or more intervening interconnect members 50 and one or more intervening bioelectrode units 40 intervening between the bioelectrode unit 40 and the controller 80. Processed electrical signals from the respective bioelectrode units 40 are output to the controller 80 by sequentially scanning the bioelectrode units 40 by, for example, the operation of the switches included in the signal processing circuits. In this manner, the controller 80 obtains the status of the muscle of the arm 90 using the myoelectric sensor 1. The controller 80 may then produce an electromyogram, for example.

When the myoelectric sensor 1 is used, the wearable portion 10 deforms in response to a muscle movement such as a muscle contraction. Since the interconnect members 50 are fixed to the wearable portion 10 by the fixing members 30, the disposition of the bioelectrode units 40 connected to the interconnect members 50 changes to conform to the deformation of the wearable portion 10. Accordingly, close adhesion between the bioelectrode unit 40 and the arm 90 is maintained. Further, the configuration having the electrode sheet 20 fixed to the wearable portion 10 reduces deformation of the electrode sheet 20, thereby reducing noise caused by deformation of the electrode sheet 20. This arrangement enables the highly accurate detection of muscle movement. For example, the S/N ratio in detection signals may be improved. Further, if a material for bonding the bioelectrode units 40 to the wearable portion 10 were used, or an adhesive material for fixing the bioelectrode units 40 to a skin were used, the contact of these materials with the skin could cause stress to the wearer, but this can be prevented by the above-described arrangement.

### Second Embodiment

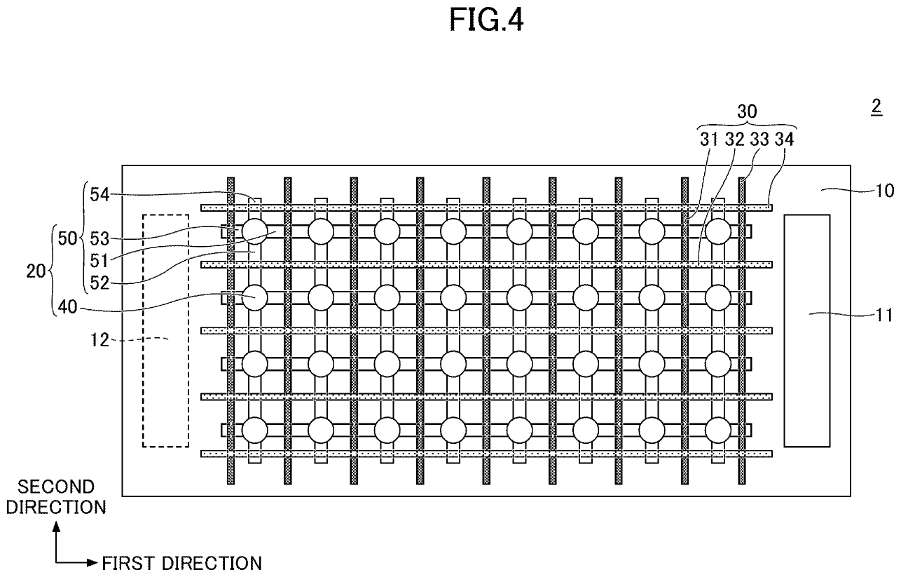

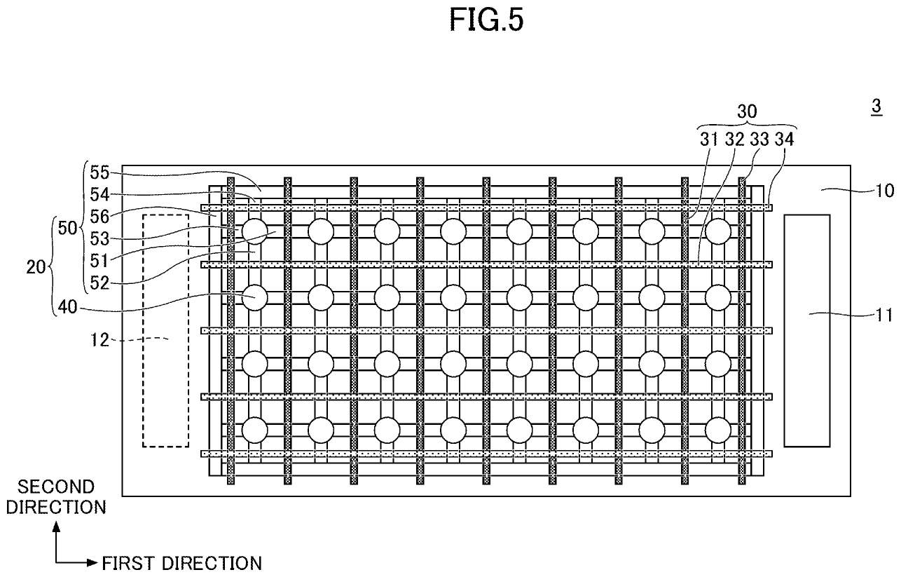

In the following, a second embodiment will be described. The second embodiment differs from the first embodiment mainly in the configuration of the electrode sheet 20 and the fixing members 30. FIG. 4 is a plan view illustrating an example of a myoelectric sensor according to the second embodiment.

A myoelectric sensor 2 according to the second embodiment includes the wearable portion 10, the electrode sheet 20, and the fixing members 30 as in the first embodiment.

In the second embodiment, the plurality of interconnect members 50 include, in addition to the plurality of first interconnect members 51 and the plurality of second interconnect members 52, a plurality of third interconnect members 53 and a plurality of fourth interconnect members 54. The third interconnect members 53 extend outwards in the first direction from the bioelectrode units 40 arranged at the outermost grid nodes in the first direction among the grid nodes constituted by the plurality of bioelectrode units 40. The fourth interconnect members 54 extend outwards in the second direction from the bioelectrode units 40 arranged at the outermost grid nodes in the second direction among the grid nodes constituted by the plurality of bioelectrode units 40. The third interconnect members 53 are arrayed in the second direction as part of the grid, and the fourth interconnect members 54 are arrayed in the first direction as part of the grid.

The fixing members 30 include third fixing members 33 and fourth fixing members 34 in addition to the first fixing members 31 and the second fixing members 32. The third fixing members 33 secure the third interconnect members 53 arrayed in the second direction to the surface 10A. The fourth fixing members 34 secure the fourth interconnect members 54 arrayed in the first direction to the surface 10A.

The remaining configurations of the second embodiment are substantially the same as those of the first embodiment.

In the second embodiment, the electrode sheet 20 is more firmly fixed to the surface 10A of the wearable portion 10. The detection accuracy is thus further improved.

In the second embodiment, the third interconnect members 53 and the fourth interconnect members 54 are provided to extend outwards from the four sides of the rectangular outline of the grid defined by the plurality of bioelectrode units 40, but may alternatively be provided to extend outwards from only one, two, or three sides of the rectangular grid outline. For example, the third interconnect members 53 may be provided to extend outwards from only an upstream side or only a downstream side of the rectangular grid outline in the first direction. Similarly, the fourth interconnect members 54 may be provided to extend outwards from only an upstream side or only a downstream side of the rectangular grid outline in the second direction. Alternatively, either only the third interconnect members 53 or only the fourth interconnect members 54 may be provided. As an example, the third interconnect members 53 may be provided only downstream of the grid in the first direction, and the fourth interconnect members 54 may be provided only downstream of the grid in the second direction, with no third interconnect member 53 provided upstream of the grid in the first direction, and no fourth interconnect member 54 provided upstream of the grid in the second direction.

### Third Embodiment

In what follows, a third embodiment will be described. The third embodiment differs from the second embodiment mainly in the configuration of the electrode sheet 20 and the fixing members 30. FIG. 5 is a plan view illustrating an example of a myoelectric sensor according to the third embodiment.

A myoelectric sensor 3 according to the third embodiment includes the wearable portion 10, the electrode sheet 20, and the fixing member 30 as in the second embodiment.

In the third embodiment, the plurality of interconnect members 50 include fifth interconnect members 55 and sixth interconnect members 56 in addition to the plurality of first interconnect members 51, the plurality of second interconnect members 52, the plurality of third interconnect members 53, and the plurality of fourth interconnect members 54. Each fifth interconnect member 55 is connected to the fourth interconnect members 54 and extends in the first direction. Each of the plurality of fourth interconnect members 54 has one end connected to a bioelectrode unit 40 and the other end connected to a fifth interconnect member 55. Each sixth interconnect member 56 is connected to the third interconnect members 53 and extends in the second direction. Each of the plurality of third interconnect members 53 has one end connected to a bioelectrode unit 40 and the other end connected to a sixth interconnect member 56.

The first fixing members 31 fix the fifth interconnect members 55 to the surface 10A in addition to fixing the plurality of first interconnect members 51. The third fixing members 33 fix the fifth interconnect members 55 to the surface 10A in addition to fixing the plurality of third interconnect members 53. The second fixing members 32 fix the sixth interconnect members 56 to the surface 10A in addition to fixing the plurality of second interconnect members 52. The fourth fixing members 34 fix the sixth interconnect members 56 to the surface 10A in addition to fixing the plurality of fourth interconnect members 54.

The remaining configurations of the third embodiment are substantially the same as those of the second embodiment.

In the third embodiment, the electrode sheet 20 is more firmly secured to the surface 10A of the wearable portion 10. The detection accuracy is thus further improved.

In the third embodiment, the fifth interconnect members 55 and the sixth interconnect members 56 are provided to surround the grid defined by the plurality of bioelectrode units 40 in plan view, but may alternatively be provided on the outside of only one, two or three sides of the grid. For example, the fifth interconnect member 55 may be provided either only upstream of or only downstream of the grid in the second direction. Similarly, the sixth interconnect member 56 may be provided either only upstream of or only downstream of the grid in the first direction. Alternatively, either only the fifth interconnect members 55 or only the sixth interconnect members 56 may be provided. When the fifth interconnect members 55 and the sixth interconnect members 56 are provided on the outside of only one, two, or three sides of the grid, no third interconnect members 53 and no fourth interconnect members 54 may be provided to extend outwards from the side where neither the fifth interconnect member 55 nor the sixth interconnect member 56 is provided. As an example, the third interconnect members 53 and the sixth interconnect member 56 may be provided only downstream of the grid in the first direction, and the fourth interconnect members 54 and the fifth interconnect member 55 may be provided only downstream of the grid in the second direction, with neither the third interconnect members 53 nor the sixth interconnect member 56 provided upstream of the grid in the first direction, and neither the fourth interconnect members 54 nor the fifth interconnect member 55 provided upstream of the grid in the second direction.

### Fourth Embodiment

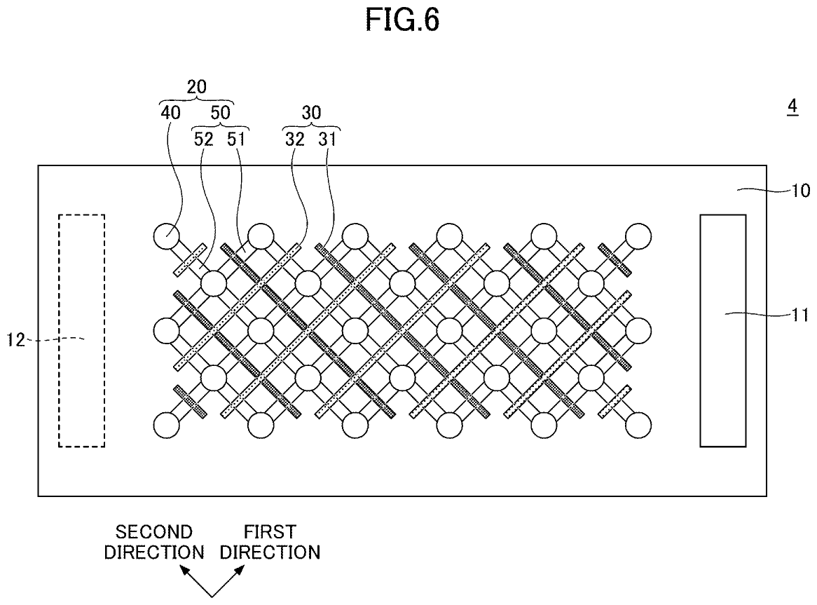

In what follows, a fourth embodiment will be described. The fourth embodiment differs from the first embodiment mainly in the configuration of the electrode sheet 20. FIG. 6 is a plan view illustrating an example of a myoelectric sensor according to the fourth embodiment.

A myoelectric sensor 4 according to the fourth embodiment includes the wearable portion 10, the electrode sheet 20, and the fixing members 30 as in the first embodiment.

In the fourth embodiment, the arrangement of the bioelectrode units 40 is defined by the first direction and the second direction that are rotated 45 degrees counterclockwise from the first direction and the second direction in the first embodiment, respectively. That is, the first direction and the second direction are at 45 degrees relative to the longitudinal direction and the transverse direction, respectively, of the wearable portion 10.

The remaining configurations of the fourth embodiment are substantially the same as those of the first embodiment.

The fourth embodiment also provide substantially the same advantageous results as those of the first embodiment.

In the myoelectric sensor 4, the electrode sheet 20 easily expands and contracts in the longitudinal and transverse directions of the wearable portion 10. As a result, when the myoelectric sensor 4 is wrapped around the arm 90 such that the transverse direction of the wearable portion 10 is substantially parallel to muscle fibers to be measured, the electrode sheet 20 easily conforms to the expansion and contraction of the muscle fibers.

In the fourth embodiment, the angle of inclination of the first and second directions relative to the longitudinal and transverse directions of the wearable portion 10 is not limited to 45 degrees, and is preferably 30 to 60 degrees, and more preferably 40 to 50 degrees.

### Fifth Embodiment

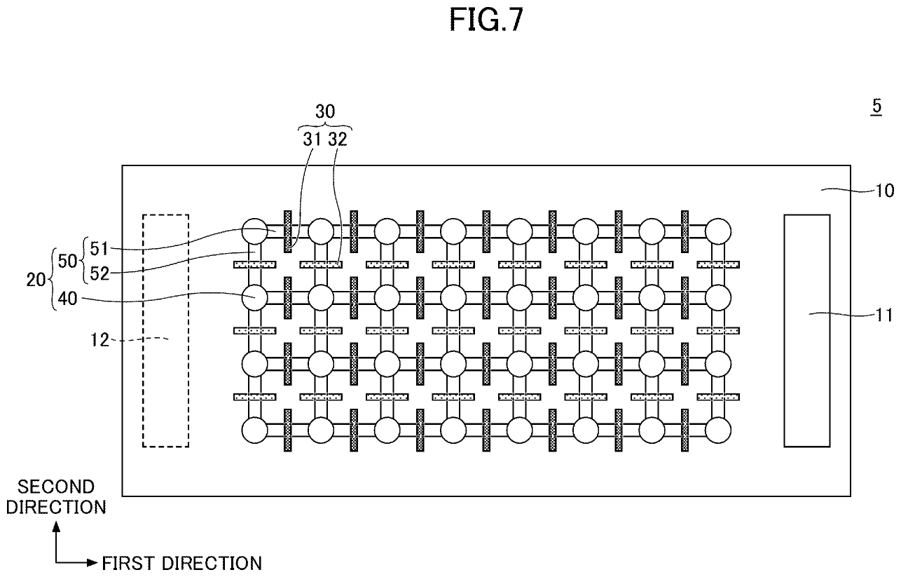

In the following, a fifth embodiment will be described. The fifth embodiment differs from the first embodiment mainly in the configuration of the fixing members 30. FIG. 7 is a plan view illustrating an example of a myoelectric sensor according to the fifth embodiment.

A myoelectric sensor 5 according to the fifth embodiment includes the wearable portion 10, the electrode sheet 20, and the fixing members 30 as in the first embodiment.

In the myoelectric sensor 5 according to the fifth embodiment, the fixing members 30 include first fixing members 31 equal in number to the first interconnect members 51 and second fixing members 32 equal in number to the second interconnect members 52. Each first fixing member 31 secures a different first interconnect member 51 to the surface 10A. Each second fixing member 32 secures a different second interconnect member 52 to the surface 10A.

The remaining configurations of the fifth embodiment are substantially the same as those of the first embodiment.

The fifth embodiment provides substantially the same advantageous results as the first embodiment.

### Sixth Embodiment

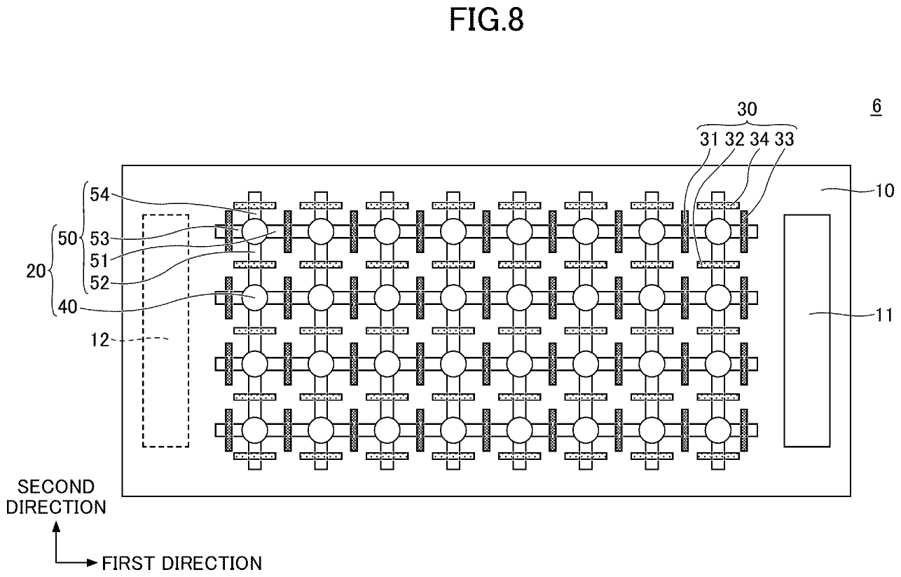

In the following, a sixth embodiment will be described. The sixth embodiment differs from the fifth embodiment mainly in the configuration of the electrode sheet 20 and the fixing members 30. FIG. 8 is a plan view illustrating an example of a myoelectric sensor according to the sixth embodiment.

A myoelectric sensor 6 according to the sixth embodiment includes the wearable portion 10, the electrode sheet 20, and the fixing members 30 as in the fifth embodiment.

As in the second embodiment, the sixth embodiment is configured such that the plurality of interconnect members 50 includes, in addition to the plurality of first interconnect members 51 and the plurality of second interconnect members 52, the plurality of third interconnect members 53 and the plurality of fourth interconnect members 54.

The fixing members 30 include, in addition to the first fixing members 31 and the second fixing members 32, third fixing members 33 equal in number to the third interconnect members 53 and fourth fixing members 34 equal in number to the fourth interconnect members 54. Each third fixing member 33 secures a different third interconnect member 53 to the surface 10A. Each fourth fixing member 34 secures a different fourth interconnect member 54 to the surface 10A.

The remaining configurations of the sixth embodiment are substantially the same as those of the fifth embodiment.

Substantially the same advantageous results as those of the second embodiment are provided by the sixth embodiment.

The fourth embodiment may alternatively be configured such that, as in the fifth embodiment, one first fixing member 31 may be provided for each first interconnect member 51, and one second fixing member 32 may be provided for each second interconnect member 52. Alternatively, the fourth embodiment may be configured such that, as in the sixth embodiment, a plurality of third interconnect members 53, a plurality of fourth interconnect members 54, third fixing members 33 equal in number to the third interconnect members 53, and fourth fixing members 34 equal in number to the fourth interconnect members 54 may be provided.

Each of the interconnect members 50 may alternatively be a cable, instead of a flexible printed circuit board.

Although the preferred embodiments and the like have heretofore been described in detail, the present invention is not limited to these embodiments and the like, and various modifications and substitutions may be made to these embodiments and the like without departing from the scope defined in the claims.

According to at least one embodiment, a myoelectric sensor with improved detection accuracy is provided.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiment(s) of the present inventions have been described in detail, it should be understood that the various changes, substitutions, and alterations could be made hereto without departing from the spirit and scope of the invention.

What is claimed is:

1. A myoelectric sensor comprising:
a wearable portion configured to be worn on a living body and deformable in response to deformation of the living body;
an electrode sheet disposed on a surface of the wearable portion; and
fixing members configured to fix the electrode sheet to the surface,
wherein the electrode sheet includes:
a plurality of bioelectrode units; and
interconnect members each electrically connecting two bioelectrode units among the plurality of bioelectrode units,
wherein the interconnect members are located between the wearable portion and the fixing members, and the fixing members press the interconnect members to the surface.

2. The myoelectric sensor as claimed in claim 1, wherein the plurality of bioelectrode units are arranged in rows and columns, each of the rows extending in a first direction, each of the columns extending in a second direction orthogonal to the first direction,
wherein the interconnect members include:
a plurality of first interconnect members each connecting bioelectrode units arranged next to each other in the first direction; and
a plurality of second interconnect members each connecting bioelectrode units arranged next to each other in the second direction, and
wherein the fixing members include:
first fixing members securing the first interconnect members to the surface; and
second fixing members securing the second interconnect members to the surface.

3. The myoelectric sensor as claimed in claim 2, wherein the first fixing members secure the first interconnect members arrayed in the second direction to the surface, and the second fixing members secure the second interconnect members arrayed in the first direction to the surface.

4. The myoelectric sensor as claimed in claim 2, wherein the interconnect members further include:
a plurality of third interconnect members extending outwards in the first direction from outermost bioelectrode units in the first direction among the plurality of bioelectrode units; and
a plurality of fourth interconnect members extending outwards in the second direction from outermost bioelectrode units in the second direction among the plurality of bioelectrode units, and
wherein the fixing members further include:
a third fixing member securing the plurality of the third interconnect members extending in the first direction to the surface; and
a fourth fixing member securing the plurality of fourth interconnect members extending in the second direction to the surface.

5. The myoelectric sensor as claimed in claim 4, wherein the third fixing member secures the plurality of third interconnect members arrayed in the second direction to the surface, and the fourth fixing member secures the plurality of fourth interconnect members arrayed in the first direction to the surface.

6. The myoelectric sensor as claimed in claim 5, further comprising:
a fifth interconnect member connected to the plurality of fourth interconnect members and extending in the first direction; and
a sixth interconnect member connected to the plurality of third interconnect members and extending in the second direction,
wherein the first fixing members and the third fixing member secure the fifth interconnect member to the surface, and the second fixing members and the fourth fixing member secure the sixth interconnect member to the surface.

7. The myoelectric sensor as claimed in claim 2, wherein the first direction and the second direction are inclined relative to a longitudinal direction and a transverse direction of the wearable portion.

8. The myoelectric sensor as claimed in claim 1, wherein the interconnect members are each a flexible printed circuit board or a cable.
